(19) United States
(12) Patent Application Publication
(10) Pub. No.: US 2023/0168259 A1
(43) Pub. Date: Jun. 1, 2023

(54) METHOD FOR PREDICTING THE COURSE OF A VIRAL DISEASE

(71) Applicant: Leibniz-Institut für Virologie, Hamburg (DE)

(72) Inventors: Gülsah Gabriel, Hamburg (DE); Stephanie Stanelle-Bertram, Hamburg (DE)

(21) Appl. No.: 17/995,728

(22) PCT Filed: Apr. 30, 2021
(86) PCT No.: PCT/EP2021/061452

§ 371 Date: Oct. 7, 2022

(30) Foreign Application Priority Data
Apr. 30, 2020 (EP) 20172395.4
Oct. 23, 2020 (EP) 20203575.4

(51) Int. Cl. G01N 33/74; A61K 31/568

(52) U.S. Cl. CPC G01N 33/743; A61K 31/568; G01N 2800/52; G01N 2800/56

(57) ABSTRACT

The invention relates to a method for predicting the course of a viral disease in a male subject infected with an influenza virus or coronavirus which is based on measuring testosterone and/or estradiol levels in said subject. The invention further relates to a method for monitoring the course of a viral disease in a male subject infected with an influenza vims or coronavirus which comprises predicting the course of the disease in said subject and assigning the subject to preventive or therapeutic measures if a severe course of said viral disease is to be expected. The invention further relates to an aromatase inhibitor for use in a method of treating or preventing a severe course of a viral disease in a male subject infected with an influenza virus or coronavirus, wherein said subject has decreased testosterone levels and/or increased estradiol levels as compared to reference values. Finally, the invention also relates to a kit for carrying out one of the aforementioned methods.

The invention relates to a method for predicting the course of a viral disease in a male subject infected with an influenza virus or coronavirus which is based on measuring testosterone and/or estradiol levels in said subject. The invention further relates to a method for monitoring the course of a viral disease in a male subject infected with an influenza virus or coronavirus which comprises predicting the course of the disease in said subject and assigning the subject to preventive or therapeutic measures if a severe course of said viral disease is to be expected. The invention further relates to an aromatase inhibitor for use in a method of treating or preventing a severe course of a viral disease in a male subject infected with an influenza virus or coronavirus, wherein said subject has decreased testosterone levels and/or increased estradiol levels as compared to reference values. Finally, the invention also relates to a kit for carrying out one of the aforementioned methods.

### FIELD OF THE INVENTION

Influenza can sometimes lead to severe disease progression with high mortality. In cases with a severe course of disease, patients may have to be treated in intensive care units (ICUs). Approximately 30% of all patients undergoing intensive care for influenza develop severe respiratory complications, in particular the acute respiratory distress syndrome (ARDS) which lead to lung failure. Severe respiratory complications can occur very rapidly in influenza patients, sometimes within only a few hours.

Similarly, ARDS is also regularly observed in a subgroup of patients which are infected with a coronavirus, in particular with the severe acute respiratory syndrome coronavirus (SARS-CoV) or severe acute respiratory syndrome coronavirus 2 (SARS-CoV-2). While about 80% of the people infected with SARS-CoV-2 recover without special treatment, about 6% of the infected people encounter severe respiratory complications, including ARDS. Elderly people and those with pre-existing conditions such as asthma, diabetes or heart disease have an increased risk of a severe course. Again, the development of severe respiratory complications can occur very fast.

So far, it is not possible to reliably predict whether a patient who is infected with an influenza virus or coronavirus will develop severe respiratory complications like ARDS. Accordingly, there is a need for new prognostic methods that enable physicians to identify patients with a particular high risk of developing a severe course of disease. Such prognostic methods would allow assigning such patients to specific treatments even before the onset of respiratory complications, thereby significantly improving their chance of survival.

### DESCRIPTION OF THE INVENTION

The studies underlying the present invention have revealed that the determination of the testosterone and/or estradiol levels in a body fluid sample of a subject, preferably in a serum sample, allows predicting whether an infectious disease which is caused by infection with an influenza virus or coronavirus takes a severe or moderate course.

Specifically, it has been found by retrospective analysis that the testosterone levels which can be detected in samples from male patients infected with an influenza virus or coronavirus are significantly lower in patients that show a severe course of disease at a later stage, including severe respiratory complications like ARDS. At the same time, the estradiol levels are higher in patients that later on show complications. In addition, it has been found herein that animals infected with SARS-CoV-2 exert an increased expression of the enzyme aromatase (also known as CYP19A1), which catalyzes the conversion of testosterone to estradiol, in the lung compared to uninfected animals. Together, these studies suggest that decreased testosterone level and/or increased estradiol level are hallmarks of an influenza virus or coronavirus infection.

Based on this insight, the present invention allows providing tests that reliably predict, based on testosterone and/or estradiol levels, whether an influenza virus or coronavirus infection takes a severe course that is likely to require intensive care measurements like artificial respiration. In this way, the methods of the invention allow an improved risk analysis in hospitals and intensive care units.

Thus, in a first aspect the present invention provides a method for predicting the course of a viral disease in a male subject infected with an influenza virus or coronavirus, said method comprising:
(a) providing a body fluid sample from the subject that is infected with said influenza virus or coronavirus;
(b) determining in said sample the concentration of testosterone and/or estradiol; and
(c) comparing the concentration obtained in step (b) with at least one testosterone and/or estradiol reference value;
   wherein the comparison of the concentration obtained in step (b) with said at least one reference value indicates whether a severe course of said viral disease is to be expected in said subject.

In step (a) of the above method, a body fluid sample obtained from the infected male subject is provided. The sample to be used in the above method can be, in principle, any type of body fluid obtained from the subject to be diagnosed. In a preferred aspect, the sample will be a blood sample, such as a whole-blood sample, or a plasma or serum sample. In an even more preferred aspect, the sample will be a serum sample, such as a human serum sample.

The sample originates from a male subject that has already been diagnosed to be infected with an influenza virus or coronavirus. The male subject can be an adult between 18 and 120 years old, but it will be preferred that the subject is at least 20 years old, at least 25 years old, at least 30 years old, at least 35 years old, at least 40 years old, at least 45 years old, at least 50 years old, at least 55 years old, or at least 60 years old.

The influenza virus or coronavirus diagnosis can be obtained from any method suitable for confirming the presence of a virus in the subject, for example by PCR-based detection of virus-specific nucleic acid, by electron microscopy, by detection of antibodies against viral proteins, or by immunodetection of viral components using conjugated antibodies, e.g. in the form of an enzyme-linked immunosorbent assay (ELISA). In a preferred aspect, the influenza virus or coronavirus diagnosis in the subject has been obtained by an ELISA.

As used herein, the term influenza virus relates to a group of RNA viruses that cause the infectious disease influenza. Common symptoms of influenza include fever, headaches, and fatigue. These symptoms are caused by large amounts of proinflammatory cytokines and chemokines that are released by influenza-infected cells, including interferon or tumor necrosis factor (TNF). It has been proposed that the massive release of cytokines can produce a life-threatening cytokine storm. The methods described herein can be used to predict such a severe course of disease. These methods can be applied to patients infected with any influenza subtype, including influenza A subtypes H1N1, H2N2, H3N2, H5N1, H7N7, H1N2, H9N2, H7N2, H7N3, H10N7, H7N9, and influenza B subtypes of the lines Victoria, Yamagata, Yamaguchi, Yokohama, Yunnan, Zhuhai.

The term coronavirus relates to a group of related viruses that cause diseases in mammals and birds. In humans, coronaviruses cause respiratory tract infections that can be linked with symptoms ranging from mild to severe. Mild infections are cause symptoms similar to those of a common cold. More severe coronavirus infections can cause life-threatening complications like the Severe Acute Respiratory Syndrome (SARS), the Middle East Respiratory Syndrome (MERS) and the Coronavirus disease 2019 (COVID-19). According to the invention, the subject can be infected with any type of a coronavirus, including viruses of the genus Alphacoronavirus, Betacoronavirus, Gammacoronavirus and Deltacoronavirus, but it will preferably be a coronavirus that is known to cause respiratory infections, such as SARS, MERS and COVID-19. It is particularly preferred that the subject is infected with the severe acute respiratory syndrome coronavirus (SARS-CoV) or the severe acute respiratory syndrome coronavirus 2 (SARS-CoV-2).

In step (b) of the above method, the concentration of testosterone and/or estradiol is determined in the sample from the infected male patient. In one embodiment, the concentration of testosterone is determined in the sample from the infected patient. Testosterone is the primary male sex hormone and plays a key role in the development of male reproductive tissues such as testes and prostate but also in regulating immune response pathways in males. Testosterone is a steroid from the androstane class which is synthesized in several steps from cholesterol. In males, testosterone is secreted primarily by the testicles. In females, which normally have testosterone levels that are 7-8 times lower compared to males, testosterone is produced in the ovaries.

Methods for determining the concentration of testosterone are well known in the art and have been described in the scientific literature, for example, in van Nuland et al. (2019), Star-Weinstock & Dey (2019), Wooding et al (2015), and Ankarberg-Lindgren at al. (2018). In addition, kits are commercially available for testosterone quantification in a sample, such as the Testosterone ELISA Assay Kit (Eagle Biosciences, Amherst, USA) or the Testosterone ELISA Kit (Abcam, Berlin, Germany).

In another embodiment, the concentration of estradiol is determined in the sample from the infected male patient. Estradiol, which is also referred to as E2 in the literature, is an estrogen steroid hormone and the major female sex hormone. As such, it is involved in the regulation of the estrous and menstrual female reproductive cycles but also in regulating immune response pathways in females. Estradiol is mandatory for development and maintenance of female reproductive tissues such as the mammary glands, uterus, and vagina during puberty, adulthood, and pregnancy. Estradiol is produced from cholesterol through a series of reactions and intermediates. In females, the production takes place especially in the follicles of the ovaries. In males, estradiol is mainly produced by catalytic conversion of testosterone, a reaction that is catalyzed by the enzyme aromatase (also known as CYP19A1).

Methods for determining the concentration of estradiol are well known in the art and have been described in the scientific literature, for example, in Wooding et al. (2015), Siqueira Ferreira et al. (2017), and Keski-Rahkonen et al. (2015), Analytical Chemistry, 87, 14, 7180-7186. In addition, kits are commercially available for estradiol quantification in a sample, such as the Estradiol Parameter Assay Kit, (R&D Systems, Inc., Minneapolis, USA), the Estradiol ELISA Kit (Eagle Biosciences, Amherst, USA) or the Human Estradiol E2 ELISA Kit (Abcam, Berlin, Germany).

It is particularly preferred that step (b) of the above method comprises the determination of both the testosterone concentration and the estradiol concentration in the sample from the infected male patient. The testosterone concentration and the estradiol concentration can be determined in the same or in different aliquots of the sample, in either order.

Once the concentration of testosterone and/or estradiol has been determined in step (b) of the above method, the concentration is compared with at least one testosterone and/or estradiol reference value. The comparison of the testosterone and/or estradiol concentration measured in the sample with at least one reference value indicates whether a severe course of said viral disease is to be expected in said subject.

In one preferred embodiment, the method of the first aspect of the invention comprises in step (b) the determination of the testosterone concentration in the body fluid sample, in particular a blood or serum sample, and step (c) comprises the comparison of the testosterone concentration of the sample with a testosterone reference value, wherein a severe course of disease is to be expected if the concentration obtained in step (b) falls below the reference value.

In males between 18-50 years, a concentration of between 8.69 to 29.00 nMol testosterone per liter blood serum is considered normal. Instead, testosterone concentrations values below 8.69 nMol/l are considered less than normal in males of that age and therefore indicative for potentially severe complication in patients infected with influenza virus or coronavirus. Therefore, in one embodiment, the reference value for adult males of that age is 8.69 nMol/l and a severe course of disease is to be expected if the concentration in the sample falls below 8.69 nMol/l. In another embodiment, the reference value for males at that age is 8.5 nMol/l and a severe course of disease is to be expected if the concentration in the sample falls below 8.5 nMol/l. In yet another embodiment, reference value for males at that age is 7.5 nMol/l and a severe course of disease is to be expected if the concentration in the sample falls below 7.5 nMol/l. In yet another embodiment, reference value for males at that age is 6.5 nMol/l and a severe course of disease is to be expected if the concentration in the sample falls below 6.5 nMol/l. In yet another embodiment, reference value for males at that age is 5.5 nMol/l and a severe course of disease is to be expected if the concentration in the sample falls below 5.5 nMol/l. In yet another embodiment, reference value for males at that age is 4.5 nMol/l and a severe course of disease is to be expected if the concentration in the sample falls below 4.5 nMol/l. In yet another embodiment, reference value for males at that age is 3.5 nMol/l and a severe course of disease is to be expected if the concentration in the sample falls below 3.5 nMol/l. In yet another embodiment, reference value for males at that age is 2.5 nMol/l and a severe course of disease is to be expected if the concentration in the sample falls below 2.5 nMol/l. In yet another embodiment, reference value for males at that age is 1.5 nMol/l and a severe course of disease is to be expected if the concentration in the sample falls below 1.5 nMol/l.

In males older than 51 years, a concentration of between 6.68 to 25.8 nMol testosterone per liter blood serum is considered normal. Instead, testosterone concentrations values below 6.68 nMol/l are considered less than normal in males of that age and therefore indicative for potentially severe complication in patients infected with influenza virus or coronavirus. Therefore, in one embodiment, the reference value for adult males of that age is 6.68 nMol/l and a severe course of disease is to be expected if the concentration in the sample falls below 6.68 nMol/l. In yet another embodiment, reference value for males at that age is 6.5 nMol/l and a severe course of disease is to be expected if the concentration in the sample falls below 6.5 nMol/l. In yet another embodiment, reference value for males at that age is 5.5 nMol/l and a severe course of disease is to be expected if the concentration in the sample falls below 5.5 nMol/l. In yet another embodiment, reference value for males at that age is 4.5 nMol/l and a severe course of disease is to be expected if the concentration in the sample falls below 4.5 nMol/l. In yet another embodiment, reference value for males at that age is 3.5 nMol/l and a severe course of disease is to be expected if the concentration in the sample falls below 3.5 nMol/l. In yet another embodiment, reference value for males at that age is 2.5 nMol/l and a severe course of disease is to be expected if the concentration in the sample falls below 2.5 nMol/l. In yet another embodiment, reference value for males at that age is 1.5 nMol/l and a severe course of disease is to be expected if the concentration in the sample falls below 1.5 nMol/l. In yet another embodiment, reference value for males at that age is 1.0 nMol/l and a severe course of disease is to be expected if the concentration in the sample falls below 1.0 nMol/l.

For estradiol, a concentration of between 27.1 and 52.2 pg estradiol per milliliter blood serum is considered normal in males, independent from their age. Instead, estradiol concentrations values above 52.2 pg/ml are considered more than normal and therefore indicative for potentially severe complication in males infected with influenza virus or coronavirus. Therefore, in one embodiment, the reference value for adult males is 52.2 pg/ml and a severe course of disease is to be expected if the estradiol concentration in the sample exceeds 52.2 pg/ml. In another embodiment, the reference value for adult males is 55 pg/ml and a severe course of disease is to be expected if the estradiol concentration in the sample exceeds 55 pg/ml. In another embodiment, the reference value for adult males is 60 pg/ml and a severe course of disease is to be expected if the estradiol concentration in the sample exceeds 60 pg/ml. In another embodiment, the reference value for adult males is 70 pg/ml and a severe course of disease is to be expected if the estradiol concentration in the sample exceeds 70 pg/ml. In yet another embodiment, the reference value for adult males is 80 pg/ml and a severe course of disease is to be expected if the estradiol concentration in the sample exceeds 80 pg/ml. In yet another embodiment, the reference value for adult males is 90 pg/ml and a severe course of disease is to be expected if the estradiol concentration in the sample exceeds 90 pg/ml. In yet another embodiment, the reference value for adult males is 100 pg/ml and a severe course of disease is to be expected if the estradiol concentration in the sample exceeds 100 pg/ml.

The above method can be used as a tool for patient surveillance. Accordingly, in a second aspect, the invention provides a method for monitoring the course of a viral disease in a male subject infected with an influenza virus or coronavirus, said method comprising:
(a) repeatedly conducting a method according to the first aspect of the invention defined above in predefined time intervals, and
(b) assigning the subject to preventive or therapeutic measures if based on the results obtained in step (a) a severe course of the viral disease is to be expected.

The predictive method according to the first aspect of the invention can be used for monitoring the course of a viral disease in a male subject infected with an influenza virus or coronavirus. Since subjects infected with influenza or coronavirus may encounter complications rather rapidly, is it helpful to predict the further course of disease in predefined time intervals, such as every 24 hours, every 18 hours, every 12 hours, or every 6 hours. A severe course of the infection with respiratory complications can be expected if a drop in the testosterone levels and/or an increase in the estradiol levels can be observed. In this case, a severe course of disease is likely and the subject can be assigned to preventive or therapeutic measures. Such measures include an increased clinical surveillance, the initiation of artificial respiration, or the administration of anti-viral drugs like remdesivir. The measures may also include the treatment of the patient with one or more aromatase inhibitors as described elsewhere herein, the treatment of the patient with one or more testosterone or testosterone derivative as described elsewhere herein, or the combinations of such therapies. In the method of the second aspect of the invention, a severe course of the viral disease may include the development of ARDS.

The observation that patients with decreased testosterone levels and/or increased estradiol levels regularly exert a higher risk to experience a severe course of diseases after infection with influenza virus or coronavirus suggests that these patients have increased amounts and/or increased activity levels of the aromatase enzyme which catalyzes the conversion of testosterone into estradiol. This is in line with the observation reported in the below Examples that the expression of aromatase is increased in hamsters infected with SARS-CoV-2 compared to uninfected animals. Accordingly, the inhibition of aromatase may have a therapeutic effect in these patients.

Thus, in a third aspect, the invention provides an aromatase inhibitor for use in a method of treating or preventing the severe course of a viral disease in a male subject infected with an influenza virus or coronavirus, wherein said subject has (a) decreased testosterone levels compared to the normal reference levels discussed above and/or (b) increased estradiol levels compared to normal reference levels discussed above.

As described in the below Examples, it was found that aromatase inhibitors can effectively block virus dissemination. Thus, in a fourth aspect, the invention provides an aromatase inhibitor for use in a method of inhibiting virus dissemination in a subject infected with an influenza virus or coronavirus.

The aromatase inhibitors referred to in the third and fourth aspect of the invention are preferably administered to a subject, more preferably a male subject, that has
(a) decreased testosterone levels, and/or
(b) increased estradiol levels
   as compared to reference values.

The aromatase inhibitor will be formulated to be compatible with the intended route of administration. Different routes of administration are feasible for providing the aromatase inhibitor to the subject. Preferably, the aromatase inhibitor is formulated for oral administration, e.g. in the form of tablets, capsules, granule, powder, liquids, and the like. Alternatively, the aromatase inhibitor can be formulated for parenteral administration, for example, for intravenous or subcutaneous administration. The aromatase inhibitor may also be formulated for being administered by implantation, e.g. by admixing the aromatase inhibitor with a three-dimensional carrier or scaffold, such as a hydrogel.

Suitable aromatase inhibitors for use herein include, but are not limited to, aminoglutethimide, testolactone, anastrozole, letrozole, exemestane, vorozole, formestane, and fadrozole. Preferably, the aromatase inhibitor is for use in a method of treating or preventing a severe course of a viral disease which includes the development of ARDS.

Preferably, the administration of an aromatase inhibitor can be combined with testosterone supplementation. Accordingly, it is particularly preferred that testosterone is administered to the subject who receives the aromatase inhibitor. Testosterone administration and administration of the aromatase inhibitor can occur simultaneously or sequentially, in either order.

In a fifth aspect, the invention provides testosterone or a testosterone derivative for use in a method of treating or preventing the severe course of a viral disease in a male subject infected with an influenza virus or coronavirus, wherein said male subject has (a) decreased testosterone levels compared to the normal reference levels discussed above and/or (b) increased estradiol levels compared to normal reference levels discussed above. The reference values will be those discussed above in connection with the method according to the first aspect of the invention.

Again, the testosterone or testosterone derivative will be formulated to be compatible with the intended route of administration. Different routes of administration are feasible for providing testosterone or its derivative to the subject. Preferably, the testosterone or testosterone derivative is formulated for oral administration, e.g. in the form of tablets, capsules, granule, powder, liquids, and the like. Alternatively, the testosterone or testosterone derivative can be formulated for parenteral administration, for example, for intravenous or subcutaneous administration. However, it is preferred that the testosterone or testosterone derivative is formulated for transdermal or transmucosal application, e.g. in the form of a patch that releases testosterone to the skin.

Preferably, as stated above, the administration of testosterone can be combined with the administration of one or more aromatase inhibitors. It is hence preferred that the subject who receives the testosterone or testosterone derivative also receives one of the aromatase inhibitors referred to above. Testosterone administration and administration of the aromatase inhibitor can occur simultaneously or sequentially, in either order.

Finally, in a sixth aspect, the invention provides a kit for carrying out the methods described herein above, comprising:
(a) means for determining whether a subject is infected with an influenza virus or coronavirus;
(b) means for determining the concentration of testosterone and/or estradiol; and
(c) optionally, buffers and diluents.

In one embodiment, the kit contains antibodies which are useful for the detection of influenza virus or coronavirus antigens, e.g. by an ELISA. The kit may also include suitable immunologic reagents for determining the concentration of testosterone and/or estradiol.

### DESCRIPTION OF THE FIGURES

FIG. 1 shows the testosterone and estradiol levels determined in a number of COVID-19 patients. (A) Table depicting the testosterone and estradiol levels measured in male and female COVID-19 patients. (B) Graphic depiction of the testosterone (a, b) and estradiol (c, d) levels were measured in the sera or plasma from COVID-19 patients and aged-matched (≥40 y) healthy controls. Male COVID-19 patients (a, c) were subdivided into patients requiring connection to an ECMO (+ECMO) and patients not being placed on ECMO (−ECMO). Bargraphs (a, c) represent males (COVID-19+ECMO, n=5; COVID-19 −ECMO, n=34; healthy controls, n=30) and bargraphs (b, d) represent females (COVID-19, n=11, healthy controls, n=20). Statistical significance was assessed by Student's t-test (* P<0.05, ** P<0.01, *** P<0.001, **** P<0.0001).

FIG. 2 shows the results from total testosterone expression level measurements in H7N9 male infected with H7N9 influenza A virus.

FIG. 3 shows the results of measuring CYP19A1 expression in the Syrian golden hamster. (a) CYP19A1 mRNA expression levels in lungs of SARS-CoV-2 infected male and female Syrian golden hamsters at 3 d p.i. (n=9-10). Relative CYP19A1 mRNA expression values in PBS treated hamsters for each sex were set to 1 after normalization against HPRT (Hypoxanthine Phosphoribosyltransferase 1). Values are shown as means and error bars are shown as SD. Statistical significance was assessed by Kruskal-Wallis one-way ANOVA followed by Dunn's multiple comparisons test (*p<0.05, ****p<0.0001). (b) CYP19A1 protein expression in lungs of SARS-CoV-2 infected male (upper panel) and female (lower panel) Syrian golden hamsters at 3 d p.i. Representative pictures for each sex (n=5) are shown. The arrowheads indicate positive signal for CYP19A1 expression.

FIG. 4 shows the results of measuring virus titer and MIP-1a/MIP-1b expression levels in different organs of hamsters treated with placebo or letrozole. (a-c) Virus titer in lungs (a), brain (b) and testis (c) of PBS and SARS-CoV-2 infected male Syrian golden hamsters treated with placebo or letrozole at 3 d p.i. (each n=6). (d-e) Protein expression levels of MIP-1a (d) and MIP-1b (e) in lungs of PBS and SARS-CoV-2 infected male Syrian golden hamsters treated with placebo or letrozole at 6 d p.i. (each n=6). (f-h) Virus titer in lungs (f), brain (g) and plasma (h) of PBS and SARS-CoV-2 infected female Syrian golden hamsters treated with placebo or letrozole at 3 d p.i. (each n=6). (i-j) Protein expression levels of MIP-1a (i) and MIP-1b (j) in lungs of PBS and SARS-CoV-2 infected female Syrian golden hamsters treated with placebo or letrozole at 6 d p.i. (each n=6). Values are shown as means and error bars are shown as SD. Statistical significance was assessed by Kruskal-Wallis one-way ANOVA followed by Dunn's multiple comparisons test (*p<0.05, ** p<0.01, *** p<0.001, **** p<0.0001). n.d. not detectable, n.s. not significant.

FIG. 5 shows the results of measuring CYP19A1 expression in the human lung of fatal Covid-19 cases. (a) CYP19A1 mRNA expression levels in lungs from fatal male Covid-19 cases and controls (non-Covid-19) who died for other reasons (non-Covid-19: n=5, Covid-19: n=9). Values are shown as means; error bars are shown as SD. Statistical significance was assessed by Kruskal-Wallis one-way ANOVA by Dunn's multiple comparisons test (*p<0.05). (b) CYP19A1 protein expression in lungs of fatal male (upper panel) and female (lower panel) Covid-19 cases or controls who died of other reasons (non-Covid-19). Detection of SARS-CoV-2 RNA by in situ hybridization. Representative pictures are shown (males: n=8, females: n=3). The squares indicate macrophages.

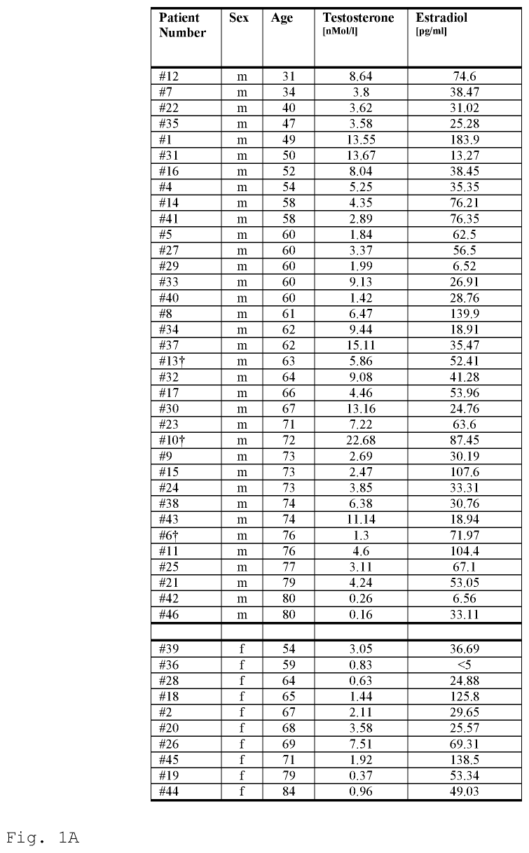

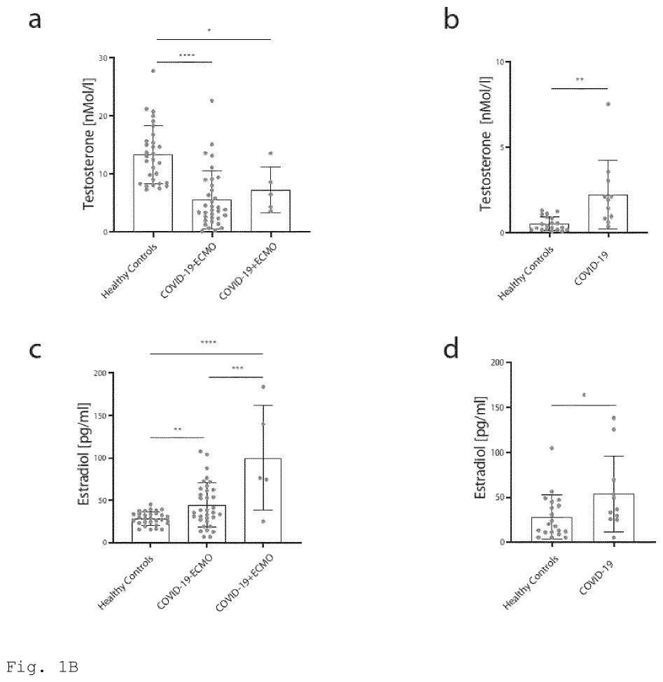

### EXAMPLES

The invention is described in the following on the basis of examples, for the purpose of illustration, without limiting the invention. It will be evident to a person skilled in the art that modifications and variations of the examples described are possible without deviating from the idea of the invention.

### Example 1: Determination of Hormone Status in COVID-19 Patients

45 COVID-19 patients at the University Hospital Hamburg Eppendorf requiring intensive care were examined. Among these patients, 35 were males and 10 were females. The median age within males and females was comparable with 62 and 67.5, respectively. Majority of the patients presented an elevated body mass index (BMI) (31.4% of males and 30% of females with a BMI ≥30). All patients presented comorbidities in males and females, such as adipositas (males 69%; females 50%), followed by diabetes type II (males 22.9%; females 20%), hypertension (males 45.7%, females 33.3%) and cancer (males 22.9%, females 33.3%). Acute respiratory distress (ARDS) detected was classified as moderate or severe in most male (37% or 26%) and female (33% or 33%) patients. Sequential organ failure assessment (SOFA) scores were evaluated in males and females presenting high (4-7) or very high (8-11) scores in males (35% or 25%) and females (40% or 60%). Due to the strong sex bias of males-to-females with a ratio of 3.5:1, sex hormones known to play a key role not only in fertility but also in innate and adaptive immunity were measured.

Results: The results are shown in Table 1. Total testosterone levels were reduced in 69% of males. Herein, 26% of males showed very low and 43% of males extremely low testosterone levels. In 60% of females, testosterone levels were increased to high (50%) or very high (10%) levels. Estradiol levels were elevated in male COVID-19 patients (46%), either to high (30%) or very high (16%) levels. Comparably, 60% of females also showed elevated estradiol concentration to high (40%) or very high (20%) levels. Thus, the vast majority of male COVID-19 patients have very low testosterone levels and very high estradiol levels. In contrast, female COVID-19 patients tend to have high testosterone and estradiol levels. A shift in sex hormones, as seen here in male patients, hints towards increased aromatase (CYP19A1) activity, i.e. the enzyme that converts testosterone to estradiol.

### Example 2: Determination of Hormone Status in H7N9 Influenza Patients

A total of n=44 avian H7N9 influenza positive cases of reproductive age (18-49 years) were enrolled with a median age of 42 years. A total of n=54 avian H7N9 influenza positive cases were included in those 50 year olds with the median age of 61 years. The male H7N9 cases accounted for 75% in the younger and 70% in the older age groups, which is consistent with previous epidemiological studies based on larger laboratory-confirmed H7N9 cohorts. Blood samples of H7N9 patients were collected within acute phases after illness onset.

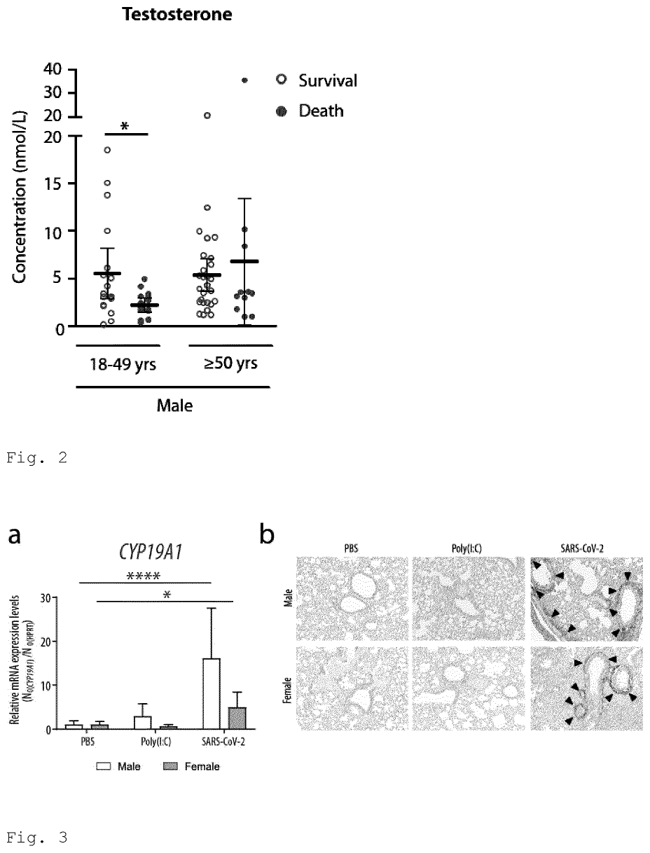

In order to assess the role of testosterone for the outcome of H7N9 infections, the testosterone concentrations were measured in all cohorts. Testosterone levels were strongly reduced in H7N9 infected men of both age groups assessed compared to virus negative H7N9 controls. Low testosterone levels strongly correlated with lethal outcome in H7N9 infected men in the age group of 18-49 year olds (P<0.001) (FIG. 2). These data show that low testosterone levels in H7N9 infected men of 18-49 years of age correlate with an enhanced risk for lethal outcome.

### Example 3: Virus Isolation and Animal Infection

The SARS-CoV-2 isolate (SARS-CoV-2/Germany/Hamburg/01/2020) was isolated by inoculation of VeroE6 cells with 200 μl of a human nasopharyngeal swab sample of a confirmed male COVID-19 patient in Hamburg, Germany and propagated for three serial passages in VeroE6 cells. VeroE6 were cultivated in DMEM (Sigma-Aldrich GmbH) with 2% fetal bovine serum, 1% penicillin-streptomycin and 1% L-glutamine at 37° C. for virus propagation and were tested negative for Mycoplasma sp. by PCR. All infection experiments with SARS-CoV-2 were performed in a biosafety level 3 (BSL-3) laboratory.

All animal experiments were performed in strict accordance with the guidelines of German animal protection law and were approved by the relevant German authority (Behörde für Gesundheit and Verbraucherschutz; protocols N 32/2020). Male and female Syrian golden hamsters (8-10 weeks old) were purchased from Janvier and were kept under standard housing conditions (21±2° C., 40-50% humidity, food and water ad libitum) with a 12:12 light-dark cycle. For infection, hamsters were anaesthetized with 150 mg/kg ketamine and 10 mg/kg xylazine by intraperitoneal injection. The animals were intranasally inoculated with 10⁵ plaque forming units (pfu) SARS-CoV-2, mock infected with PBS or were administered with 1 mg kg-1 Poly(I:C). On day 3 p.i., five animals per group were euthanized by intraperitoneal injection of an overdosis of pentobarbital, and blood was drawn by cardiac puncture.

For RNA isolation, the lungs were stored in RNAprotect Tissue Reagent (QIAGEN). For histopathological examinations the collected lungs were fixed by immersion in 10% neutral-buffered formalin and embedded in paraffin.

### Example 4: Determination of CYP19A1 mRNA Expression

For the determination of CYP19A1 mRNA expression levels by real-time quantitative PCR (RT-qPCR), RNAprotect-fixed lungs from hamsters were homogenized in 700 μl lysis buffer RL with 5 sterile, stainless steel beads (diameter 2 mm, Retsch) at 30 Hz and 4° C. for 10 min in the mixer mill MM400 (Retsch). Total RNA was isolated from homogenized lung supernatants using the innuPREP RNA Mini Kit 2.0 (Analytik Jena) according to the manufacturer's instructions with an additional on column DNase I treatment using the RNase-free DNase Set (QIAGEN). The RNA was eluted in RNase-free water and mixed with 1 U μl-1 RiboLock RNase inhibitor (Thermo Fisher Scientific). For cDNA synthesis random nonamer primers (Gene Link, pd(N)9, final concentration: 5 μM) and the SuperScript III Reverse Transcriptase (Thermo Fisher Scientific) were used according to the manufacturer's instructions, using 2 pg total RNA.

The cDNA was generated using the GeneAmp PCR System 9700 (Applied Biosystems; cycle: 25° C. for 5 min, 50° C. for 60 min, 70° C. for 15 min, 4° C. hold). Reactions were set up with PCR grade water (Roche Life Science) in LightCycler® 480 Multi-well Plate 96 Reaction Plate (Roche Life Science). Briefly, 2 μl of cDNA template were added to 10 μl FastStart Essential DNA Green Master (Roche Life Science) and 300 nM of forward and reverse primer. RT-qPCR runs were conducted on the LightCycler® 96 Real-Time PCR System (Roche Life Science) with endpoint fluorescence detection: 10 min at 95° C. and 45 amplification cycles (15s at 95° C., 10s at 65° C. and 20s at 72° C.) Analysis was performed in duplicate for CYP19A1 and reference gene (hamster: HPRT, human: RPL32) in each sample. Negative controls and samples without reverse transcriptase were included to detect contaminations.

Relative expression values were determined using a modified E^{-ΔΔCt} method. Rn-values were exported from the LightCycler® 96 Software v1.1.0.1320 (Roche) to Microsoft Office Excel 2016 and N₀-value for the starting concentration of the transcript in the original sample were obtained with LinReg PCR Software v2018.0 (Ruijter et al. 2009). The averaged N₀-value of the CYP19A1 gene was then normalized with the average N₀-value for HPRT (N_{0(HPRT)}) or RPL32 (N_{0(RPL32)}) of the respective sample. The relative N_{0(CYP19A1)}/N_{0(HPRT)}- or N_{0(CYP19A1)}/N_{0(RPL32)}-expression values of the biological replicates are presented.

The following primer sequences were used for qRT-PCR of HPRT1 (hypoxanthin-guanin-phosphoribosyltransferase 1) and CYP19A1 in the hamster lung:

HPRT1 forward
(SEQ ID NO: 1)
5′-TCCCAGCGTCGTGATTAGTG-3′
HPRT1 reverse
(SEQ ID NO: 2)
5′-GTGATGGCCTCCCATCTCTT-3′
CYP19A1 forward
(SEQ ID NO: 3)
5′- ATGCGGCACATCATGCTGAA-3′
CYP19A1 reverse
(SEQ ID NO: 4)
5′- TCTTTCAAGTCCTTGGCGGAT-3′

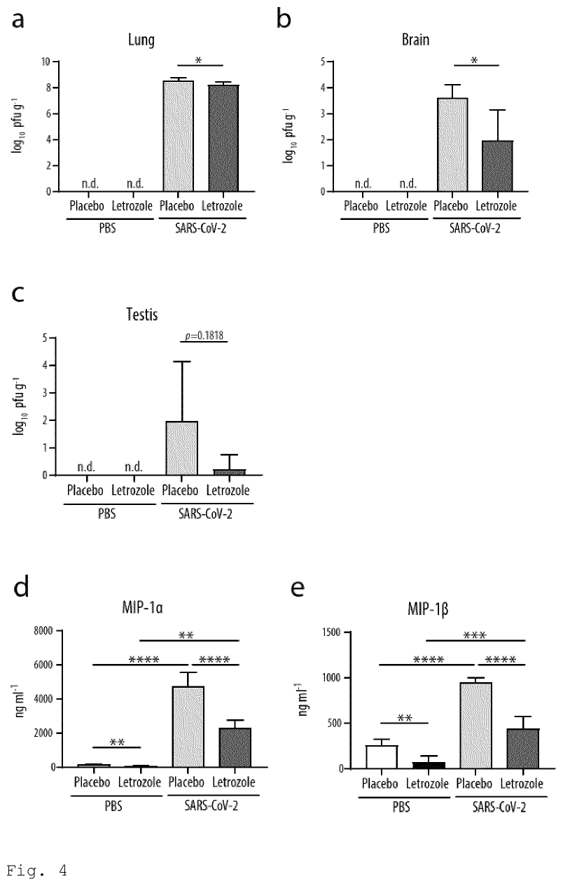

Results: The results are shown in FIG. 3(a). It can be seen that the aromatase expression as determined by RT-qPCR is significantly higher in animals infected with SARS-CoV-2 compared to non-infected animals.

### Example 5: Immunohistochemistry

For immunohistochemical detection of aromatase, the EnVision+ System (Dako Agilent Pathology Solutions) was used. Serial sections of tissue were dewaxed and rehydrated in isopropanol and 96% ethanol followed by blockage of endogenous peroxidase by incubation in 85% ethanol with 0.5% H₂O₂ for 30 min at room temperature. Antigen retrieval was performed by incubation in citrate buffer (10 mM citric acid, 0.05% Tween 20) for 20 min in a microwave at 800 W, followed by 20 min at room temperature. Sections were afterwards transferred to Shandon Coverplates™ (Thermo Electron GmbH) and stained with a polyclonal antibody directed against aromatase (Abcam, ab18995) diluted 1:500 in PBS containing 1% BSA, 0.3% Triton X-100 over night at 4° C. Sections were subsequently rinsed, and the peroxidase-labeled polymer was applied as secondary antibody for 30 minutes. Visualization of the reaction was accomplished by incubation in chromogen 3,3-diaminobenzidine tetrahydrochloride (DAB, 0.05%) and 0.03% H₂O₂ in PBS for 5 min and afterwards counterstained with Mayer's hematoxylin for 1 min. For negative controls, the primary antibody was replaced by rabbit normal serum (1:3,000).

Results: The results are shown in FIG. 3(b). It can be seen that aromatase protein expression can be detected in lungs of SARS-CoV-2-infected male (upper panel) and female (lower panel) hamsters. No aromatase protein expression can be detected in the lungs of control animals.

### Example 6: Letrozole Treatment

All animal experiments were performed in strict accordance with the guidelines of German animal protection law and were approved by the relevant German authority (Behörde für Gesundheit and Verbraucherschutz; protocols N 103/2020). Male and female Syrian golden hamsters (8-12 weeks old) were purchased from Janvier or bread at the Heinrich Pette Institute (Leibniz Institute for Experimental Virology, Hamburg, Germany) and were kept under standard housing conditions (21±2° C., 40-50% humidity, food and water ad libitum) with a 16:8 light-dark cycle. For infection, hamsters were anaesthetized with 150 mg/kg ketamine and 10 mg/kg xylazine by intraperitoneal injection. The animals were intranasally inoculated with 10⁵ plaque forming units (p.f.u.) SARS-CoV-2 or mock infected with PBS. At 3 hours and each following day p.i., animals were treated with 0.18 mg kg-1 letrozole or placebo by intraperitoneal injection. On day 3 and 6 p.i., six animals per group were euthanized by intraperitoneal injection of an overdose of pentobarbital and blood was drawn by cardiac puncture. For virus titer determination and cytokine measurements, lungs, brains and testis were collected, homogenized in 1 ml 1×PBS and stored at −80° C.

Homogenization of organs was performed in 1 ml 1×PBS with 5 sterile, stainless steel beads (Ø 2 mm, Retsch) at 30 Hz for 10 min in the mixer mill MM400 (Retsch). The plaque assays were performed on VeroE6 cell monolayers and stained with crystal violet after 72 hours. The tissue homogenisates were titrated on VeroE6 cells in 10-fold serial dilutions for 30 min at 37° C. and overlaid with MEM (Sigma-Aldrich) supplemented with 0.2% BSA, 1% L-glutamine, 1% penicillin-streptomycin, 1 μg ml-1 L-1-tosylamido-2-phenylethyl chloromethyl ketone (TPCK) treated trypsin (Sigma-Aldrich) and 1.25% Avicel. After 72 hours p.i., cells were fixed with 4% paraformaldehyde and the plaques were visualized by crystal violet staining.

Protein expression levels of macrophage inflammatory protein 1α and 1β (MIP-1α, MIP-1β) were measured in homogenized lungs using a custom-made Bio-Plex Prom Mouse Cytokine multiplex (Bio-Rad) in a Bio-Plex 200 System with high-throughput fluidics (HTF; Bio-Rad) according to the instructions provided by the manufacturer.

All data were analysed with Prism software (GraphPad, 9.0.1) using Kruskal-Wallis one-way analysis of variance (ANOVA) followed by Dunn's multiple comparisons test. Statistical significance was defined as p<0.05 (*p<0.05, ** p<0.01, *** p<0.001, **** p<0.0001).

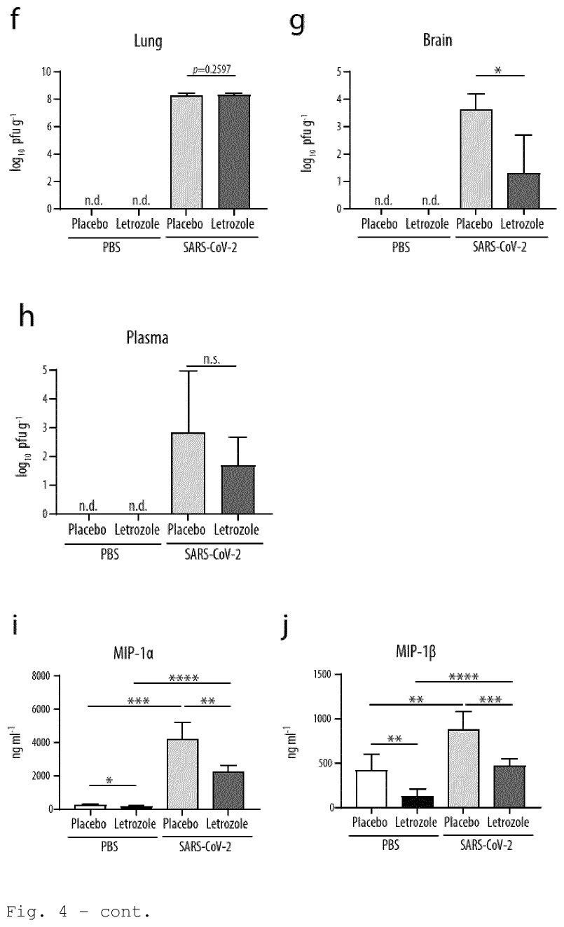

Results: The results are shown in FIG. 4. It can be taken from FIGS. 4 (a)-(c) that treatment with the aromatase inhibitor letrozole results in lower virus titers in the lungs, brain and testis in SARS-CoV-2-infected male animals compared to placebo. Similarly, FIGS. 4 (f)-(h) demonstrate that treatment with the aromatase inhibitor letrozole results in lower virus titers in the lungs, brain and plasma in SARS-CoV-2-infected female animals compared to placebo. These data suggest that the aromatase inhibitor may inhibit virus dissemination. FIG. 4 further shows that the expression levels MIP-1α and MIP-1β are lower in the lungs of animals treated with letrozole both in male (d)-(e) and female (i)-(j) animals.

### Example 7: CYP19A1 Expression in Lung Autopsies of Men with Fatal Covid-19

It was analyzed whether the data derived from preclinical animal model are also reflected in humans. Therefore, autopsy material from the lungs of men and women who died of Covid-19 (n=54) was analyzed. As controls, lung material obtained from men and women who died for other reasons (non-Covid-19 control group) was analyzed as well. Pathological assessment was performed at three independent study sites: in Hamburg (n=26 males, n=8 females), in Tübingen (n=8 males, n=3 females) and in Rotterdam (n=12 males, n=1 female).

Total RNA from formalin-fixed, paraffin-embedded human lung tissue sections was purified using the RNeasy® FFPE Kit (Qiagen) according to the manufacturer's instructions. To detect SARS-CoV-2 RNA in lung tissue, in situ hybridization (ISH) was performed by hybridizing lung tissue sections using specific probes for SARS-CoV-2 (ACD, Newark, Calif., USA) followed by the RNAscope 2.5 HD Detection Kit Red from ACD (Newark, Calif., USA) according to the manufacturer's protocol.

qRT-PCR was performed as described in Example 4 above, wherein The following primer sequences were used for qRT-PCR of RPL32 (Ribosomal Protein L32) and CYP19A1 in the human lung:

RPL32 forward
(SEQ ID NO: 5)
5′- GAAGTTCCTGGTCCACAACG-3′
RPL32 reverse
(SEQ ID NO: 6)
5′ -GCGATCTCGGCACAGTAAG-3′
CYP19A1 forward
(SEQ ID NO: 7)
5′ -CGGCCTTGTTCGTATGGTCA-3′
CYP19A1 reverse
(SEQ ID NO: 8)
5′- CAGAAGGGTCAACACGTCCA-3′

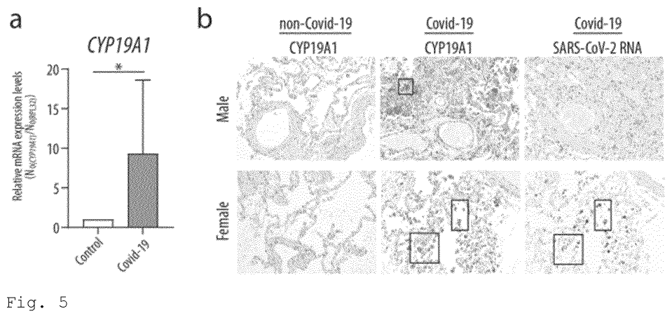

Results: At all sites, CYP19A1 was abundantly expressed in the lungs of Covid-19 males compared to non-Covid-19 male controls. In general, CYP19A was expressed in epithelial cells, in endothelial cells but most profoundly in macrophages at all three study sites independently. Noteworthy, SARS-CoV-2 NP protein or RNA was still detectable in the lungs of most deceased females, while viral antigen or RNA was expressed at low levels or was already cleared at the time point of death in males. Quantification of CYP19A1 mRNA levels revealed a transcriptional increase up to ˜10-times in the lungs of Covid-19 males compared to non-Covid-19 males. These findings show that CYP19A1 is also abundantly expressed at the time point of death in the lungs of men with Covid-19. The result are depicted in FIG. 5.

### LITERATURE

1. Ankarberg-Lindgren et al. (2018), J Steroid Biochem Mol Biol, 183: 116-124.
2. Siqueira Ferreira et al. (2017), Journal of Chromatography B 1064, 109-114.
3. Star-Weinstock & Dey (2019), Clinical Mass Spectrometry, 13, 27-35.
4. Wooding et al (2015), Steroids, 96:89-94,
5. Van Nuland et al. (2019), J Pharm Biomed Anal., 170: 161-168.

1. Method for predicting the course of a viral disease in a male subject infected with an influenza virus or coronavirus, said method comprising:
(a) providing a body fluid sample from the subject that is infected with said influenza virus or coronavirus;
(b) determining in said sample the concentration of testosterone and/or estradiol; and
(c) comparing the concentration obtained in step (b) with at least one testosterone and/or estradiol reference value;
wherein the comparison of the concentration obtained in step (b) with said at least one reference value indicates whether a severe course of said viral disease is to be expected in said subject.

2. Method according to claim 1, wherein said body fluid sample is a blood, plasma or serum sample.

3. Method according to claim 1, wherein said method comprises the comparison of the concentration obtained in step (b) with a testosterone reference value, wherein a severe course of said viral disease is to be expected if the concentration obtained in step (b) falls below the reference value.

4. Method according to claim 1, wherein said testosterone reference value is 8.69 nMol/l blood serum in males between 18-50 years or 6.68 nMol/l blood serum in males older than 51 years.

5. Method according to claim 1, wherein said method comprises the comparison of the concentration obtained in step (b) with an estradiol reference value, wherein a severe course of said viral disease is to be expected if the concentration obtained in step (b) exceeds the reference value.

6. Method according to claim 1, wherein said estradiol reference value is 52.2 pg/ml blood serum, more preferably 60 pg/ml blood serum.

7. Method according to claim 1, wherein said method comprises:
(i) the comparison of the testosterone concentration obtained in step (b) with a testosterone reference value, wherein said reference value preferably is 8.69 nMol/l blood serum in males between 18-50 years or 6.68 nMol/l blood serum in males older than 51 years, and
(ii) the comparison of the estradiol concentration obtained in step (b) with an estradiol reference value, wherein said reference value preferably is 52.2 pg/ml blood serum,
wherein a severe course of said viral disease is to be expected if the testosterone concentration obtained in step (b) falls below the testosterone reference value and the estradiol concentration obtained in step (b) exceeds the estradiol reference value.

8. Method according to claim 1, wherein said influenza virus is H7N9 or said coronavirus is SARS-CoV-2.

9. A method for monitoring the course of a viral disease in a subject infected with an influenza virus or coronavirus, said method comprising:
(a) repeatedly conducting a method as defined in claim 1 in predefined time intervals, and
(b) assigning the subject to preventive or therapeutic measures if based on the results obtained in step (a) a severe course of said viral disease is to be expected.

10. Method according to claim 1, wherein said severe course of the viral disease includes the development of the acute respiratory distress syndrome (ARDS).

11. A method of treating or preventing a severe course of a viral disease in a subject infected with an influenza virus or coronavirus, the method comprising administering to the subject an aromatase inhibitor, wherein said subject has
(a) decreased testosterone levels, and/or
(b) increased estradiol levels
as compared to reference values prior to said administering step.

12. The method of claim 11, wherein said inhibitor is selected from the group consisting of aminoglutethimide, testolactone, anastrozole, letrozole, exemestane, vorozole, formestane, and fadrozole.

13. The method of claim 11, wherein said severe course of the viral disease includes the development of the acute respiratory distress syndrome (ARDS).

14. The method of claim 11, wherein said method further comprises administering testosterone or a testosterone derivative to said subject.

15. A method of inhibiting virus dissemination in a subject infected with an influenza virus or coronavirus, said method comprising administering an aromatase inhibitor to said subject.

16. The method of claim 15, wherein said subject has
(a) decreased testosterone levels, and/or
(b) increased estradiol levels
as compared to reference values.

17. The method of claim 15, wherein said inhibitor is selected from the group consisting of aminoglutethimide, testolactone, anastrozole, letrozole, exemestane, vorozole, formestane, and fadrozole.

18. The method of claim 15, wherein said subject is a male subject.

19. A method of treating or preventing a severe course of a viral disease in a male subject infected with an influenza virus or coronavirus, said method comprising administering testosterone or a testosterone derivative to said male subject, wherein said male subject has (a) decreased testosterone levels compared to the normal reference levels and/or (b) increased estradiol levels compared to normal reference levels prior to said administering step.

20. Kit for carrying out the method of claim 1, comprising:
(a) means for determining whether a subject is infected with an influenza virus or coronavirus;
(b) means for determining the concentration of testosterone and/or estradiol; and
(c) optionally, buffers and diluents.


SEQUENCE LISTING (8 sequences)

SEQ ID NO: 1 - DNA, 20 bp, Artificial Sequence
  primer sequence
tcccagcgtcgtgattagtg

SEQ ID NO: 2 - DNA, 20 bp, Artificial Sequence
  primer sequence
gtgatggcctcccatctctt

SEQ ID NO: 3 - DNA, 20 bp, Artificial Sequence
  primer sequence
atgcggcacatcatgctgaa

SEQ ID NO: 4 - DNA, 21 bp, Artificial Sequence
  primer sequence
tctttcaagtccttggcggat

SEQ ID NO: 5 - DNA, 20 bp, Artificial Sequence
  primer sequence
gaagttcctggtccacaacg

SEQ ID NO: 6 - DNA, 19 bp, Artificial Sequence
  primer sequence
gcgatctcggcacagtaag

SEQ ID NO: 7 - DNA, 20 bp, Artificial Sequence
  primer sequence
cggccttgttcgtatggtca

SEQ ID NO: 8 - DNA, 20 bp, Artificial Sequence
  primer sequence
cagaagggtcaacacgtcca